(19) United States
(12) Patent Application Publication
(10) Pub. No.: US 2020/0003717 A1
(43) Pub. Date: Jan. 2, 2020

(54) GAS SENSOR AND METHOD OF MANUFACTURING THE SAME

(71) Applicant: National Taiwan University of Science and Technology, Taipei City (TW)

(72) Inventors: Jinn P. CHU, Taipei City (TW); Bohr-Ran HUANG, Taipei City (TW); Markos Mehretie YENESEW, Taipei City (TW)

(21) Appl. No.: 16/353,275

(22) Filed: Mar. 14, 2019

Related U.S. Application Data
(60) Provisional application No. 62/691,116, filed on Jun. 28, 2018.

(51) Int. Cl. G01N 27/12; H01L 29/06; H01L 29/22; H01L 21/02; G01N 33/00

(52) U.S. Cl. CPC G01N 27/127; H01L 29/0676; H01L 29/22; H01L 21/02425; H01L 21/02444; G01N 33/0047; H01L 21/02606; H01L 21/02645; G01N 33/005; G01N 33/0054; H01L 21/02554

(57) ABSTRACT

A gas sensor includes a substrate, a thin film metallic glass, an ultrananocrystalline diamond layer and a sensor structure. The thin film metallic glass is formed on the substrate. The ultrananocrystalline diamond layer partially covers the thin film metallic glass. The sensor structure includes a seed layer formed on the ultrananocrystalline diamond layer and a plurality of nanostructures formed on the seed layer.

### CROSS-REFERENCE TO RELATED APPLICATION

This application claims the priority benefits of U.S. provisional application Ser. No. 62/691,116, filed on Jun. 28, 2018, the entirety of which is hereby incorporated by reference herein and made a part of this specification.

### BACKGROUND OF THE INVENTION

### 1. Field of the Invention

The present disclosure generally relates to a gas sensor, and more particularly to a gas sensor with an ultrananocrystalline diamond material. The present disclosure further comprises a method of manufacturing the gas sensor.

### 2. Description of Related Art

Gas sensors are used to sense specific gases in the atmosphere, such as hydrogen, oxygen, carbon dioxide or other harmful gases. Most of gasses are colorless and odorless and cannot be directly recognized by the human eye or sense of smell, so it is necessary to sense these gases with gas sensors. The sensing principle of the gas sensor is that it mainly detects a change in an electrical parameter (such as voltage, current, resistance, or conductivity) when sensing a specific gas, and then the gas sensor monitors the degree of change to determine the concentration of the specific gas.

At present, gas sensors can be classified into the semiconductor type, the catalytic combustion type, the electrochemical type and the like according to different designs. For example, a main sensor structure of a semiconductor gas sensor is mostly made of a metal oxide (such as zinc oxide, tin oxide, etc.), and a sensing signal is derived by sensing a resistance change in the main sensor structure after the gas is adsorbed by the main sensor structure. However, the high resistivity and limited operating temperature range of metal oxides may affect the reaction time and sensitivity of the gas sensor, and the gas sensing selectivity of the gas sensor may be limited.

Therefore, there is a need to provide a gas sensor that obviates the above problems.

### SUMMARY OF THE INVENTION

A primary object of this disclosure is to provide a gas sensor having improved sensor response effectively by combining an ultrananocrystalline diamond material with a metallic glass material.

To achieve the aforesaid and other objects, the gas sensor includes a substrate, a thin film metallic glass, an ultrananocrystalline diamond layer and a sensor structure. The thin film metallic glass is formed on the substrate. The ultrananocrystalline diamond layer partially covers the thin film metallic glass. The sensor structure includes a seed layer formed on the ultrananocrystalline diamond layer and a plurality of nanostructures formed on the seed layer.

In one embodiment of this disclosure, a coverage of the ultrananocrystalline diamond layer on the thin film metallic glass is 50% to 90%.

In one embodiment of this disclosure, the thin film metallic glass comprises a copper-based thin film metallic glass or a silver-based thin film metallic glass.

In one embodiment of this disclosure, each nanostructure is configured as a zinc oxide nanotube or a zinc oxide nanorod.

In one embodiment of this disclosure, the gas sensor further comprises an electrode layer formed on the sensor structure.

In one embodiment of this disclosure, the gas sensor is capable of sensing the gas at ambient temperature.

In one embodiment of this disclosure, the gas sensor is a hydrogen gas sensor, an ammonia sensor, or an acetone sensor.

In one embodiment of this disclosure, when the gas is hydrogen gas, a detectable concentration range of hydrogen gas of the gas sensor is from 10 ppm to 500 ppm, and a sensitivity of the gas sensor is above 34%.

In one embodiment of this disclosure, when the concentration of the hydrogen gas is 100 ppm, a decay in sensitivity of the gas sensor is less than 1% for 60 days.

The method of manufacturing the gas sensor of this disclosure comprises: providing a substrate; forming a thin film metallic glass on the substrate; depositing an ultrananocrystalline diamond layer on the thin film metallic glass, wherein the ultrananocrystalline diamond layer partially covers the thin film metallic glass; and forming a sensor structure on the ultrananocrystalline diamond layer.

In one embodiment of this disclosure, the method further comprises: forming an electrode layer on the sensor structure.

In one embodiment of this disclosure, a coverage of the ultrananocrystalline diamond layer on the thin film metallic glass is adjustable by controlling a deposition time of the ultrananocrystalline diamond layer.

### BRIEF DESCRIPTION OF THE DRAWINGS

The accompanying drawings are included to provide further understanding of the invention and are incorporated in and constitute a part of this specification. The drawings illustrate embodiments of the invention and, together with the descriptions, serve to explain the principles of the invention.

FIG. 1 illustrates a structural configuration of the gas sensor of this disclosure;

FIG. 2 illustrates a flowchart of a method of manufacturing the gas sensor of this disclosure;

FIG. 3 illustrates a structural configuration corresponding to each step of the method of manufacturing the gas sensor of this disclosure;

FIG. 4 illustrates the relationships of the concentration of hydrogen gas and sensor response measured from the gas sensor of this disclosure and different comparative examples;

FIG. 5 illustrates photoluminescence spectra measured from the gas sensor of this disclosure and different comparative examples;

FIG. 6 illustrates the response curve of the gas sensor of this disclosure exposed to a hydrogen atmosphere;

FIG. 7 illustrates sensor responses of the gas sensor of this disclosure exposed to different gaseous atmospheres;

FIG. 8 illustrates the response curve of the gas sensor of this disclosure exposed to a hydrogen atmosphere for a period of time; and

FIG. 9 illustrates the response curve of the gas sensor of this disclosure used for sensing hydrogen gas for a longer period of time.

### DESCRIPTION OF THE EMBODIMENTS

Since various aspects and embodiments are merely exemplary and not limiting, after reading this specification, skilled artisans will appreciate that other aspects and embodiments are possible without departing from the scope of the disclosure. Other features and benefits of any one or more of the embodiments will be apparent from the following detailed description and the claims.

As used herein, the terms “comprises,” “comprising,” “includes,” “including,” “has,” “having” or any other variation thereof are intended to cover a non-exclusive inclusion. For example, a component, structure, article, or apparatus that comprises a list of elements is not necessarily limited to only those elements but may include other elements not expressly listed or inherent to such component, structure, article, or apparatus.

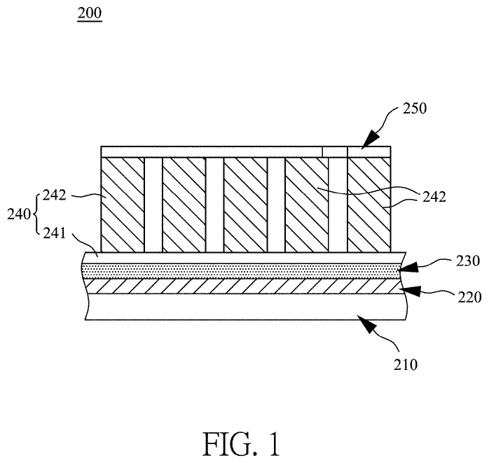

Refer to FIG. 1, which illustrates a structural configuration of a gas sensor of this disclosure. As illustrated in FIG. 1, the gas sensor 200 of this disclosure comprises a substrate 210, a thin film metallic glass 220, an ultrananocrystalline diamond layer 230 and a sensor structure 240.

In one embodiment of this disclosure, the substrate 210 may comprise a silicon wafer, but the substrate 210 may also comprise a III-V semiconductor, glass, quartz, sapphire or the like. The substrate 210 may further comprise plastic or other polymer materials. The material of the substrate 210 is selected depending on different requirements, but this disclosure is not limited thereto.

The thin film metallic glass 220 is formed on the substrate 210; that is, the thin film metallic glass 220 is formed on one side of the substrate 210. The thin film metallic glass 220 has an amorphous structure, in which the atoms are arranged irregularly or without specific order in the structure. The thin film metallic glass 220 has several satisfactory properties including minimum grain boundary defects, excellent mechanical properties, low electron scattering and low current leakage. In one embodiment of this disclosure, the thin film metallic glass 220 comprises a copper-based thin film metallic glass or a silver-based thin film metallic glass, but this disclosure is not limited thereto. For example, the copper-based thin film metallic glass may include copper, zirconium, aluminium and titanium, such as a Cu—Zr—Al—Ti alloy (Cu: 47 at %, Zr: 42 at %, Al: 7 at % and Ti: 4 at %), but the copper-based thin film metallic glass may also comprise other alloys, and this disclosure is not limited thereto. In one embodiment of this disclosure, the thickness of the thin film metallic glass 220 is about 5-20 nm, but this disclosure is not limited thereto.

The ultrananocrystalline diamond layer 230 is formed on the thin film metallic glass 220; that is, the ultrananocrystalline diamond layer 230 is formed on the side of the thin film metallic glass 220 opposite to the side of the thin film metallic glass 220 connected to the substrate 210. The ultrananocrystalline diamond layer 230 is a thin film layer on one side of the thin film metallic glass 220. The ultrananocrystalline diamond layer 230 is made of an ultrananocrystalline diamond material, and the ultrananocrystalline diamond layer 230 partially covers the thin film metallic glass 220. The ultrananocrystalline diamond layer 230 comprises a nanoscale structural defect, such that the thin film metallic glass 220 cannot be covered fully by the ultrananocrystalline diamond layer 230. The nanoscale structural defect may be formed by the breakage of a sp3-bond of diamond caused by hydrogen during the grain growth process of the ultrananocrystalline diamond layer 230. For example, the ultrananocrystalline diamond layer 230 is composed of a plurality of very fine grains. The size of each grain is about 3-5 nm and the grains have different atomic arrangements from one another, such that a nanoscale structural defect is formed between the adjacent two grains and is a so-called grain boundary. The ultrananocrystalline diamond layer 230 has a very high grain boundary concentration HO %) because of the grain boundaries formed by the plurality of grains, but this disclosure is not limited thereto. For example, the nanoscale structural defect may also comprise a dislocation between adjacent two grains or a defect of another type. In one embodiment of this disclosure, the thickness of the ultrananocrystalline diamond layer 230 is about 10-50 nm, but this disclosure is not limited thereto.

Furthermore, in one embodiment of this disclosure, a coverage of the ultrananocrystalline diamond layer 230 on the thin film metallic glass 220 is about 50% to 90%, but this disclosure is not limited thereto. The combination of the substrate 210, the thin film metallic glass 220 and the ultrananocrystalline diamond layer 230 serves as the base of the gas sensor 200 and provides a preferred forming environment for the sensor structure 240. Because of the structural configuration of the thin film metallic glass 220 and the ultrananocrystalline diamond layer 230, in conjunction with the nanoscale structural defect of the ultrananocrystalline diamond layer 230, a better electron capturing effect may be provided by the ultrananocrystalline diamond layer 230 to increase the conductivity; additionally, the life of the charge carrier may be extended and the carrier transfer time may be reduced. Accordingly, the sensor performance of the gas sensor 200 of this disclosure may be enhanced effectively.

The sensor structure 240 is formed on the ultrananocrystalline diamond layer 230; that is, the sensor structure 240 is formed on the side of the ultrananocrystalline diamond layer 230 opposite to the side of the ultrananocrystalline diamond layer 230 connected to the thin film metallic glass 220. The sensor structure 240 comprises a seed layer 241 and a plurality of nanostructures 242. The seed layer 241 is formed on the ultrananocrystalline diamond layer 230, and the plurality of nanostructures 242 are formed on the seed layer 241. In one embodiment of this disclosure, the sensor structure 240 comprises a metal oxide, such as zinc oxide, which has the advantages of low cost, no toxicity, high stability, and the like, but the invention is not limited thereto.

The plurality of nanostructures 242 are arranged on the seed layer 241, and a gap is formed between any two adjacent nanostructures 242. In one embodiment of this disclosure, each nanostructure 242 is configured as a zinc oxide nanotube or a zinc oxide nanorod. The zinc oxide nanotube or the zinc oxide nanorod may cause more defects than other materials due to the oxygen vacancies of the zinc oxide, such that each nanostructure 242 may provide a better electron capture capability. Each nanostructure 242 is substantially vertical to the surface of the seed layer 241, and one side of each nanostructure 242 is connected to the seed layer 241. In one embodiment of this disclosure, the average diameter of the nanostructures 242 is about 100 nm, and the height of each nanostructure 242 is about 2-3 p.m.

Accordingly, the sensor structure 240 may provide a larger sensing area via the disposal of the plurality of nanostructures 242, and the gas may pass through the gaps between the plurality of nanostructures 242 and be adsorbed easily by the surfaces of the plurality of nanostructures 242 for sensing.

In addition, in one embodiment of this disclosure, the gas sensor 200 further comprises an electrode layer 250. The electrode layer 250 is formed on the sensor structure 240. For example, the electrode layer 250 is formed on the side of each nanostructure 242 opposite to the side of each nanostructure 242 connected to the seed layer 241, but the position of the electrode layer 250 may also be changed according to different requirements. Here, the electrode layer 250 comprises an interdigitated electrode, but the invention is not limited thereto.

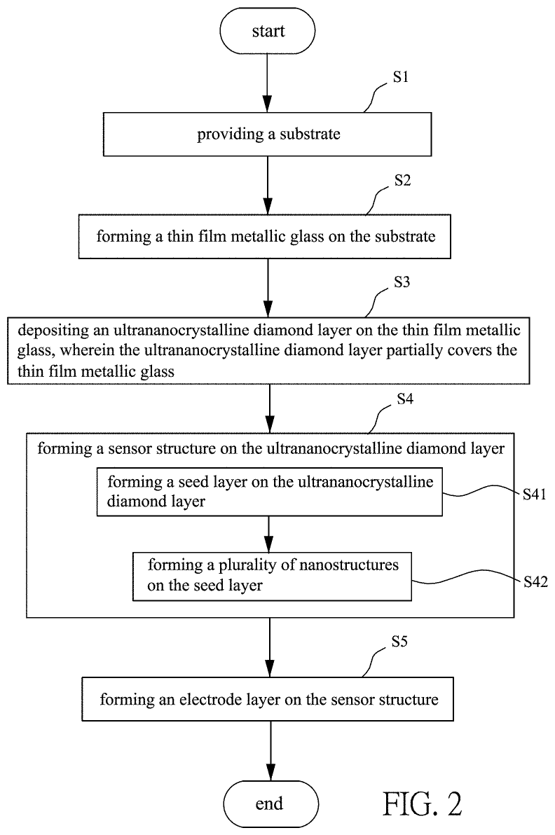

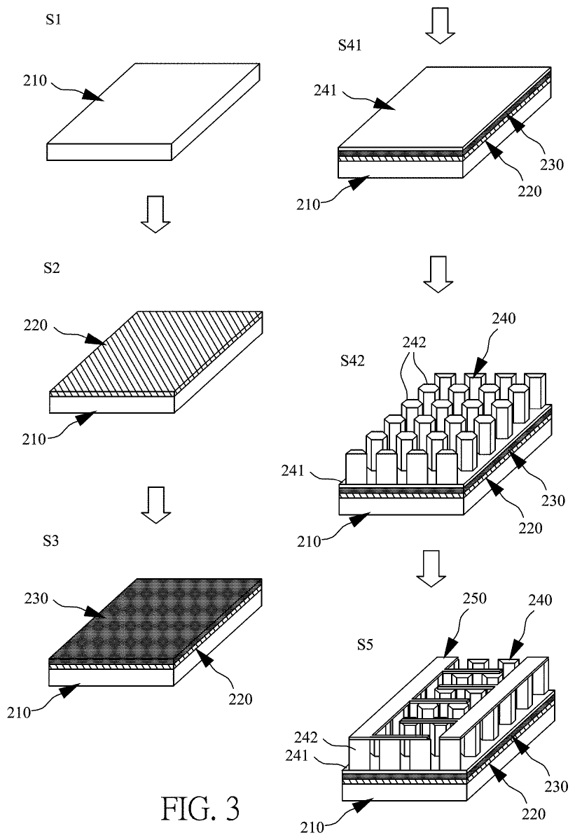

Please refer to FIG. 2 and FIG. 3. FIG. 2 illustrates a flowchart of a method of manufacturing the gas sensor of this disclosure, and FIG. 3 illustrates a structural configuration corresponding to each step of the method of manufacturing the gas sensor of this disclosure. As illustrated in FIG. 2 and FIG. 3, the method of manufacturing the gas sensor of this disclosure comprises Steps S1 to S4, which are described in detail below.

Step S1: Providing a substrate.

First, a suitable substrate 210 is provided according to the use requirements of the gas sensor 200 of this disclosure. Here, the substrate 210 may be a prepared sheet or block material having a fixed size. In this disclosure, the substrate 210 is a silicon wafer, but this disclosure is not limited thereto. A cleaning and drying process may be performed on the silicon wafer serving as the substrate 210 so as to remove dust or organic contaminants on the surface.

Step S2: Forming a thin film metallic glass on the substrate.

After the substrate 210 has been provided in Step S1, the thin film metallic glass 220 is formed on one side of the substrate 210. In one embodiment of this disclosure, a target made of the metallic glass material (for example, in one embodiment of this disclosure, a Cu₄₇Zr₄₂Al₇Ti₄ alloy target) is used to sputter the thin film metallic glass 220 on the side of the substrate 210 by a radio frequency magnetron sputtering process. The radio frequency magnetron sputtering process is performed by using a radio frequency magnetron sputtering system for the side of the substrate 210 with the target made of the metallic glass material, and the operating conditions for the sputtering system are a base pressure of about 1.0*10⁻⁶ mTorr, a working pressure of about 3 mTorr, and a sputtering distance of about 10 cm in an argon atmosphere. The argon is flowed into the environment of the sputtering process at a fluid velocity of about 20 sccm. In addition, the substrate 210 may be rotated at a rotation speed of about 20 rpm during the sputtering process to maintain a sputtering uniformity of the thin film metallic glass 220 on the substrate 210.

Step S3: Depositing an ultrananocrystalline diamond layer on the thin film metallic glass, wherein the ultrananocrystalline diamond layer partially covers the thin film metallic glass.

After the thin film metallic glass 220 has been formed in Step S2, the ultrananocrystalline diamond layer 230 is deposited on the thin film metallic glass 220. Another cleaning and drying process may be performed on the substrate 210 on which the thin film metallic glass 220 has been formed so as to remove dust or organic contaminants on the surface. In one embodiment of this disclosure, a plasma composed of a mixed gas of hydrogen, methane and argon (for example, in one embodiment of this disclosure, the plasma is composed of 2% hydrogen, 8% methane and 90% argon) is used to deposit an ultrananocrystalline diamond thin film as the ultrananocrystalline diamond layer 230 on the surface of the thin film metallic glass 220 by a microwave plasma-enhanced chemical vapor deposition (MPECVD) process. The microwave plasma-enhanced chemical vapor deposition process is performed by using a microwave plasma-enhanced chemical vapor deposition system for depositing the ultrananocrystalline diamond layer 230, and the operating conditions for the deposition system are a pressure of about 60 Torr, a plasma microwave power of about 1200 W, and a deposition time of about 7.5 minutes.

Because the ultrananocrystalline diamond layer 230 only partially covers the thin film metallic glass 220 (in one embodiment of this disclosure, the coverage of the ultrananocrystalline diamond layer 230 on the thin film metallic glass 220 is about 50% to 90%), in Step S3, the coverage of the ultrananocrystalline diamond layer 230 on the thin film metallic glass 220 is adjustable by controlling a deposition time of the ultrananocrystalline diamond layer 230. For example, the deposition time is about 5-10 minutes, but this disclosure is not limited thereto.

Step S4: Forming a sensor structure on the ultrananocrystalline diamond layer.

After the ultrananocrystalline diamond layer 230 has been provided in Step S3, the sensor structure 240 is formed on the ultrananocrystalline diamond layer 230. In one embodiment of this disclosure, Step S4 further comprises Step S41 and Step S42, which are described in detail below.

Step S41: Forming a seed layer on the ultrananocrystalline diamond layer.

After the ultrananocrystalline diamond layer 230 has been provided in Step S3, the seed layer 241 of the sensor structure 240 is formed on the ultrananocrystalline diamond layer 230. In one embodiment of this disclosure, the seed layer 241 is formed by using a seed layer material comprising zinc oxide on the surface of the ultrananocrystalline diamond layer 230 (i.e., on the side of the ultrananocrystalline diamond layer 230 opposite to the side of the ultrananocrystalline diamond layer 230 connected to the thin film metallic glass 220) by spin coating. To increase the crystallinity of the seed layer 241, an annealing process may be performed on the formed seed layer 241. The annealing process is performed by placing the seed layer 241 at a fixed temperature of 350° C. for a period of time under a nitrogen atmosphere. However, the fixed temperature and time required for the annealing process may be adjusted according to different requirements, and the invention is not limited thereto.

Step S42: Forming a plurality of nanostructures on the seed layer.

After the seed layer 241 has been provided in Step S41, the plurality of nanostructures 242 of the sensor structure 240 are formed on the surface of the seed layer 241 (i.e., on the side of the seed layer 241 opposite to the side of the seed layer 241 connected to the ultrananocrystalline diamond layer 230). In one embodiment of this disclosure, the semi-finished product of the gas sensor 200 comprising a seed layer 241 already formed is immersed in an equimolar solution in deionized water which comprises zinc acetate (Zn(CH₃COO₂).2H₂O) and hexamethyltetramine (C₆H₁₂N₄) and is stood at a constant temperature of about 95° C. for about 3 hours, such that zinc oxide is deposited on the surface of the seed layer 241 by a hydrothermal method to form a plurality of nanostructures 242. Each of the plurality of nanostructures 242 may be configured as a nanorod or a nanotube independently connected to the seed layer 241, and one end of each nanostructure 241 is an open end opposite to the other end of each nanostructure 241 connected to the seed layer 241.

As illustrated in FIG. 2 and FIG. 3 again, in this embodiment, the method of manufacturing the gas sensor of this disclosure further comprises Step S5: Forming an electrode layer on the sensor structure.

After the sensor structure 240 has been formed in Step S4, the electrode layer 250 is formed on the sensor structure 240. In one embodiment of this disclosure, the electrode layer 250 may be an interdigitated electrode produced by sputtering a platinum target on the plurality of nanostructures 242 of the sensor structure 240 (i.e., the open ends of the plurality of nanostructures 242 not connected to the seed layer 241).

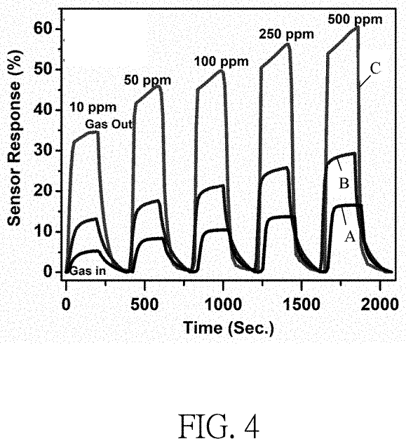

Please refer to FIG. 4, which illustrates the relationships of the concentration of the hydrogen gas and sensor response measured from the gas sensor of this disclosure and different comparative examples. In the following descriptions, the gas sensor 200 of this disclosure serves as the experimental example and other gas sensors with different conditions serve as the comparative examples. Gas measurements were respectively performed for the experimental example and the comparative examples under the same environment settings, and the data of the experimental example and the comparative examples were compared so as to confirm the actual efficacy of the gas sensor 200 of this disclosure. In the following experiments, the gas to be sensed was supplied to the gas sensors of the experimental example and each control example under the normal ambient temperature condition (i.e., room temperature) so as to measure and record the relevant parameters for comparison.

In one embodiment, the sensor structures of the gas sensors in the experimental example and the comparative examples were all made of zinc oxide as the main material, and each nanostructure was configured as a nanorod. The gas sensor without the thin film metallic glass and the ultrananocrystalline diamond layer (i.e., the gas sensor in which the sensor structure 240 is formed directly on the substrate 210) is referred to as the comparative example A, the gas sensor without the ultrananocrystalline diamond layer (i.e., the gas sensor in which the sensor structure 240 is formed directly on the surface of the thin film metallic glass 220 formed on the substrate 210) is referred to as the comparative example B, and the gas sensor 200 of this disclosure is referred to as the experimental example C. The comparative examples A and B and the experimental example C were respectively exposed to different hydrogen concentrations (in ppm) under the same environmental conditions to measure and calculate the sensor response (in %) of each gas sensor, as shown in Table 1.

TABLE 1

| | hydrogen concentration | | | | |
|---|---|---|---|---|---|
| | 10 ppm | 50 ppm | 100 ppm | 250 ppm | 500 ppm |
| comparative | 5.3 | 8.4 | 10.4 | 13.7 | 16.5 |
| example A | | | | | |
| comparative | 13.2 | 17.6 | 21.4 | 25.8 | 29.3 |
| example B | | | | | |
| experimental | 34.5 | 45.9 | 49.6 | 56.2 | 60.5 |
| example C | | | | | |

The sensor response of the gas sensor may be calculated by measuring the change in resistance during the sensing process caused by adsorption or desorption of gas molecules on the surface of the sensor structure of the gas sensor. The calculation formula of the sensor response is as follows:

S=(Rv−Rg)/Rv×100%

where S is the sensor response, Rv is the electrical resistance value in the absence of hydrogen gas, and Rg is the electrical resistance value in the presence of hydrogen gas. Generally, when the gas sensor has adsorbed gas molecules, the electrical resistance value may be decreased. Therefore, if the value of the sensor response is higher, the sensing effect of the gas sensor is better.

As shown in FIG. 4 and Table 1, in the range of hydrogen concentration from 10 ppm to 500 ppm, the sensor responses of the gas sensors for all of the comparative examples A and B and the experimental example C increased with increases in hydrogen concentration. It is obvious that regardless of the hydrogen concentration, the experimental example C demonstrated a sensor response better than those of the comparative examples A and B. Even when exposed to atmospheres with very low hydrogen concentrations (e.g., 10 ppm), experimental example C may maintain a sensor response greater than 34%. In addition, as shown in FIG. 4, a sensitivity (which is defined as the slope of the response curve of the gas sensor) exhibited by the experimental example C also exceeds the sensitivity exhibited by the comparative examples A and B. Accordingly, the gas sensor 200 of this disclosure may provide a better hydrogen sensing effect by the structural combination and arrangement of the thin film metallic glass and the ultrananocrystalline diamond layer.

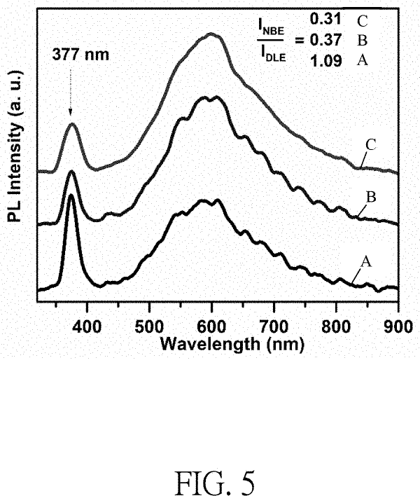

Refer to FIG. 5, which illustrates photoluminescence spectra measured from the gas sensor of this disclosure and different comparative examples. Here, light beams such as ultraviolet light were used to respectively irradiate the comparative examples A and B and the experimental example C under the same environmental settings to perform photoluminescence intensity tests. As illustrated in FIG. 5, the spectral results for all of the comparative examples A and B and the experimental example C showed two prominent emission regions. The first emission was generated at a wavelength of about 377 nm. The first emission may be attributed to the near-band edge emission (NBE) associated with band-to-band excitonic recombination in zinc oxide. The second emission was generated at a wavelength of about 500-700 nm. The second emission may be attributed to the deep-level emission (DLE) caused by the recombination of photo-generated charges with crystal defects of zinc oxide. Comparing the maximum photoluminescence intensity I of the NBE and the DLE of each examples, it can be found that the I_{NBE}/I_{DLE} of the comparative example A was about 1.09, the I_{NBE}/I_{DLE} of the comparative example B was about 0.37, and the I_{NBE}/I_{DLE} of the experimental example C was about 0.31. In other words, compared with the comparative examples A and B, the zinc oxide sensor structure of the experimental example C may have formed more defects. Accordingly, the number of defects in the zinc oxide sensor structure of the gas sensor 200 of this disclosure may be increased by the structural combination and arrangement of the thin film metallic glass and the ultrananocrystalline diamond layer so as to enhance the electron capture capability and the conductivity and thereby improve the sensing effect of hydrogen.

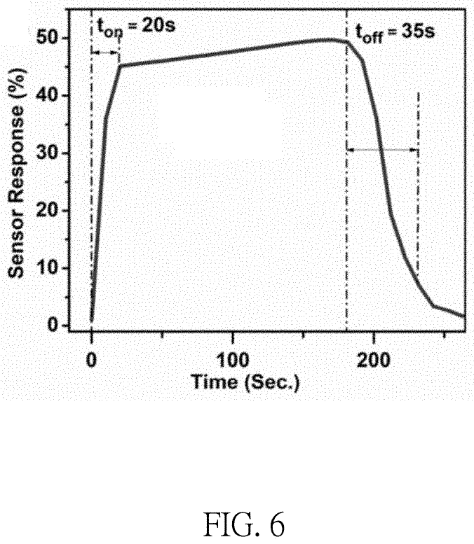

A response time (in seconds) and a recovery time (in seconds) are also important parameters for a gas sensor. Please refer to FIG. 6, which illustrates the response curve of the gas sensor of this disclosure exposed to hydrogen atmosphere. The gas sensor 200 of this disclosure was exposed to an atmosphere having a hydrogen concentration of 100 ppm under normal ambient temperature to measure and calculate the changes in sensor response over time. As illustrated in FIG. 6, under the aforementioned hydrogen atmosphere, the gas sensor 200 of this disclosure exhibited a response time Tₒₙ of about 20 seconds and a recovery time T_{off} of about 35 seconds. After the recovery time, the response of the gas sensor 200 of this disclosure recovered about 90% of the response.

Further, according to the prior arts and the aforementioned experimental data, the sensor parameters of sensor response, response time, recovery time, and operating temperature for gas sensors made of different materials and the gas sensor 200 of this disclosure exposed to an atmosphere having a hydrogen concentration of 100 ppm are shown in Table 2, respectively. Therefore, the differences between the gas sensor 200 of this disclosure and the known gas sensors may be compared. Most known gas sensors comprise a sensor structure having nanorods or nanotubes on the substrate formed by using zinc oxide or a similar metal oxide as the main material, and different materials may be added to form gas sensors.

TABLE 2

| | Sensor | Response | Recovery | Operating |
|---|---|---|---|---|
| | response | time | time | temperature |
| Sensor material | (%) | (s) | (s) | (° C.) |
| ZnO tubes | 16.2 | — | — | RT |
| ZNRs-Pd | 9 | 108 | 122 | RT |
| (Pd nanoparticles | | | | |
| attached to ZnO | | | | |
| nanorods) | | | | |
| ZnO-ITO | 18 | — | — | RT |
| (ZnO deposited on | | | | |
| ITO) | | | | |
| ZNR/In | 20.5 | — | — | RT |
| (ZnO nanorods | | | | |
| deposited on In) | | | | |
| CuO | 3.72 | — | — | 250 |
| ZnO/Al | 10 | 600 | — | 100 |
| (ZnO deposited on Al) | | | | |
| ZNRs/AC | 23.2 | 18 | 15 | RT |
| (ZnO nanorods | | | | |
| covered by activated | | | | |
| carbon) | | | | |
| ZnO-MWCNT | 1.5 | — | 25 | RT |
| (ZnO deposited on | | | | |
| MWCNT) | | | | |
| ZNT-graphene | 28.1 | 30 | 38 | RT |
| (ZnO nanotubes | | | | |
| deposited on graphene) | | | | |
| CNT-Pd—Ni | 7.5 | 312 | 150 | RT |
| (Pd/carbon nanotube/ | | | | |
| Ni multilayer | | | | |
| structure) | | | | |
| ZNR | 10.4 | — | — | RT |
| (ZnO nanorods, | | | | |
| comparative | | | | |
| example A) | | | | |
| ZNR/UNCD/TFMG | 49.6 | 20 | 35 | RT |
| (ZnO nanorods | | | | |
| deposited on thin film | | | | |
| metallic glass and | | | | |
| ultrananocrystalline | | | | |
| diamond layer, | | | | |
| experimental example | | | | |
| C) | | | | |

As shown in Table 2, except for a few gas sensors that need to operate at higher specific operating temperatures, most gas sensors may be used for sensing at room temperature (RT). Sensor response, response time and recovery time vary with different sensor materials and structural configuration. However, it is obvious that the sensor response of the gas sensor 200 of this disclosure is better than those of the known gas sensors, and the performances on response time and recovery time of the gas sensor 200 of this disclosure are equally outstanding. Accordingly, it is sufficient to demonstrate that the gas sensor 200 of this disclosure may exhibit better sensor response and related sensing parameters because of the structural combination and configuration of the thin film metallic glass and the ultrananocrystalline diamond layer.

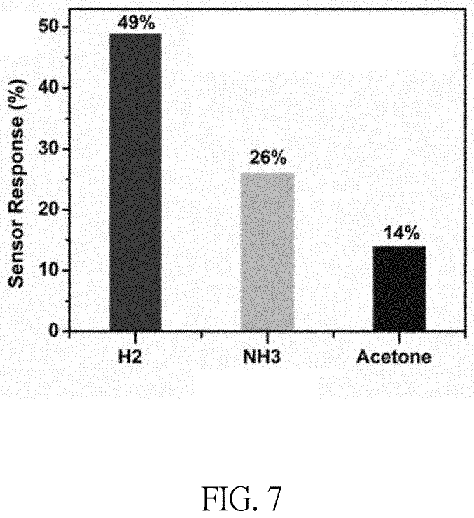

In addition, selectivity to gases is another important parameter for a gas sensor. Please refer to FIG. 7, which illustrates sensor responses of the gas sensor of this disclosure exposed to different gaseous atmospheres. The gas sensor 200 of this disclosure was exposed respectively to atmospheres having hydrogen, ammonia, and acetone concentrations of 100 ppm under normal ambient temperature to measure and calculate the sensor response. As illustrated in FIG. 7, the gas sensor 200 of this disclosure has good sensor response performance to hydrogen, ammonia and acetone, such that the gas sensor 200 of this disclosure may be used as a hydrogen sensor, an ammonia gas sensor or an acetone sensor according to different requirements. The sensor response of the gas sensor 200 of this disclosure for hydrogen is higher than the sensor response of the gas sensor 200 of this disclosure for ammonia or acetone, such that the gas sensor 200 of this disclosure has a high selectivity to hydrogen.

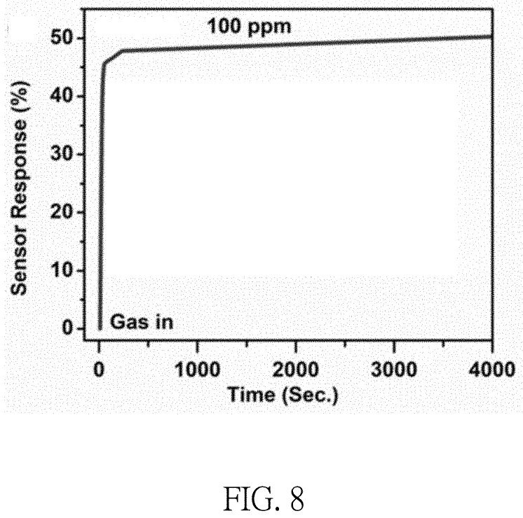

The ability to produce signals and the long-term stability of sensing for continuous exposure to a gaseous atmosphere are also important parameters for gas sensors. Please refer to FIG. 8, which illustrates the response curve of the gas sensor of this disclosure exposed to a hydrogen atmosphere for a period of time. The gas sensor 200 of this disclosure was continuously exposed to an atmosphere having a hydrogen concentration of 100 ppm for more than one hour under normal ambient temperature to measure and calculate the changes in sensor response over time. As illustrated in FIG. 8, when hydrogen gas was continuously introduced, hydrogen molecules were continuously adsorbed by the surface of the sensor structure of the gas sensor 200 of this disclosure, which greatly reduced the resistance value to a stable state, and the sensor response in terms of response time substantially rose and then slowly increased. Accordingly, a better ability to produce signals may be maintained even if the gas sensor 200 of this disclosure is continuously exposed to a gaseous atmosphere for a certain period of time.

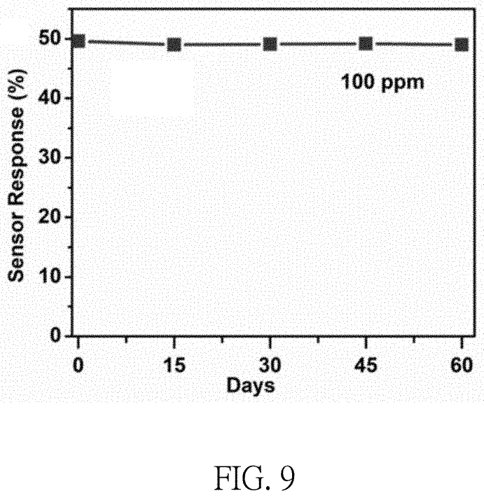

Please refer to FIG. 9, which illustrates the response curve of the gas sensor of this disclosure used for sensing hydrogen gas for a longer period of time. The gas sensor 200 of this disclosure was exposed to an atmosphere having a hydrogen concentration of 100 ppm for 60 days at normal ambient temperature to repeatedly measure and calculate the sensor response every 15 days. As illustrated in FIG. 9, the gas sensor 200 of this disclosure may exhibit a better sensor response after each sensing; even after 60 days, a decay in sensitivity of the gas sensor of the gas sensor 200 of this disclosure is less than 1%. In other words, the sensor response of the gas sensor 200 of this disclosure after performing hydrogen sensing for 60 days is almost the same as the sensor response of the gas sensor 200 of this disclosure after the initial sensing. Accordingly, the gas sensor 200 of this disclosure may maintain a better gas sensing effect even after long-term use of the gas sensor 200.

In summary, the gas sensor of this disclosure is based on the structural combination and configuration of a thin film metallic glass and an ultrananocrystalline diamond layer, and the sensor response of the gas sensor and related sensing parameters may be improved by the material properties of the metallic glass and the ultrananocrystalline diamond and the formed sensor structure to provide better gas sensing effects. Further, when the gas sensor of this disclosure is applied to sensing of a gas such as hydrogen gas, ammonia gas or acetone, the effect is remarkable.

The above detailed description is merely illustrative in nature and is not intended to limit the embodiments of the subject matter or the application and uses of such embodiments. Moreover, while at least one exemplary embodiment has been presented in the foregoing detailed description, it should be appreciated that a vast number of variations exist. It should also be appreciated that the exemplary one or more embodiments described herein are not intended to limit the scope, applicability, or configuration of the claimed subject matter in any way. Rather, the foregoing detailed description will provide those skilled in the art with a convenient guide for implementing the described one or more embodiments. Also, various changes can be made to the function and arrangement of elements without departing from the scope defined by the claims, which include known equivalents and foreseeable equivalents at the time of filing this patent application.

What is claimed is:

1. A gas sensor, comprising:
a substrate;
a thin film metallic glass formed on the substrate;
an ultrananocrystalline diamond layer partially covering the thin film metallic glass; and
a sensor structure, comprising a seed layer formed on the ultrananocrystalline diamond layer and a plurality of nanostructures formed on the seed layer.

2. The gas sensor of claim 1, wherein a coverage of the ultrananocrystalline diamond layer on the thin film metallic glass is 50% to 90%.

3. The gas sensor of claim 1, wherein the thin film metallic glass comprises a copper-based thin film metallic glass or a silver-based thin film metallic glass.

4. The gas sensor of claim 1, wherein each nanostructure is configured as a zinc oxide nanotube or a zinc oxide nanorod.

5. The gas sensor of claim 1, further comprising an electrode layer formed on the sensor structure.

6. The gas sensor of claim 1, wherein the gas sensor is capable of sensing a gas at ambient temperature.

7. The gas sensor of claim 6, which is a hydrogen gas sensor, an ammonia sensor, or an acetone sensor.

8. The gas sensor of claim 6, wherein when the gas is hydrogen gas, a detectable concentration range of hydrogen gas of the gas sensor is from 10 ppm to 500 ppm, and a sensitivity of the gas sensor is above 34%.

9. The gas sensor of claim 8, wherein when the concentration of the hydrogen gas is 100 ppm, a decay in sensitivity of the gas sensor is less than 1% for 60 days.

10. A method of manufacturing a gas sensor, comprising:
providing a substrate;
forming a thin film metallic glass on the substrate;
depositing an ultrananocrystalline diamond layer on the thin film metallic glass, wherein the ultrananocrystalline diamond layer partially covers the thin film metallic glass; and
forming a sensor structure on the ultrananocrystalline diamond layer.

11. The method of claim 10, further comprising: forming an electrode layer on the sensor structure.

12. The method of claim 10, wherein a coverage of the ultrananocrystalline diamond layer on the thin film metallic glass is adjustable by controlling a deposition time of the ultrananocrystalline diamond layer.
